(19) United States
(12) Patent Application Publication
(10) Pub. No.: US 2003/0087237 A1
(43) Pub. Date: May 8, 2003

(54) Low-temperature cycle extension of DNA with high polymerization specificity

(76) Inventors: Guofan Hong, Shanghai (CN); Yongjie Yang, Shanghai (CN); Jia Zhu, Shanghai (CN)

Correspondence Address: Marlana Titus, Nash & Titus, LLC, Suite 1000, 3415 Brookeville Road, Brookeville, MD, 20833, US

(21) Appl. No.: 09/878,131

(22) Filed: Jun. 8, 2001

(30) Foreign Application Priority Data
Apr. 30, 2001 (CN) 01117603.2.

(51) Int. Cl. C12Q 1/68; C12P 19/34; C12N 9/22

(52) U.S. Cl. 435/6; 435/91.2; 435/199

(57) ABSTRACT

The invention relates to methods for extending a primer or a pair of primers in low-temperature cycle DNA amplification for cycle sequencing and PCR. In particular, the methods contemplate the combined use of moderately thermostable DNA polymerases in the presence of a low concentration of glycerol or ethylene glycol, or the mixtures thereof, as an agent to reduce the melting temperature of DNA (that is, the temperature at which the double-strands of DNA are denatured). Predistributed reaction mixtures of a high-fidelity and high processivity DNA polymerase stable at room temperature for several weeks in ready-to-use kits are also contemplated by the invention.

### BACKGROUND OF THE INVENTION

The genetic material of all known living organisms is deoxyribonucleic acid (DNA), except in certain viruses whose genetic material may be ribonucleic acid (RNA). DNA consists of a chain of individual deoxynucleotides chemically linked in specific sequences. Each deoxynucleotide contains one of the four nitrogenous bases which may be adenine (A), cytosine (C), guanine (G) or thymine (T), and a deoxyribose, which is a pentose, with a hydroxyl group attached to its 3′ position and a phosphate group attached to its 5′ position. The contiguous deoxynucleotides that form the DNA chain are connected to each other by a phosphodiester bond linking the 5′ position of one pentose ring to the 3′ position of the next pentose ring in such a manner that the beginning of the DNA molecule always has a phosphate group attached to the 5′ carbon of a deoxyribose. The end of the DNA molecule always has an OH (hydroxyl) group on the 3′ carbon of a deoxyribose.

DNA usually exists as a double-stranded molecule in which two antiparallel DNA strands are held together by hydrogen bonds between the bases of the individual nucleotides of the two DNA strands in a strictly matched “A-T” and “C-G” pairing manner. It is the order or sequence of the bases in a strand of DNA that determines a gene which in turn determines the type of protein to be synthesized. Therefore, the accurate determination of the sequence of the bases in a DNA strand which also constitutes the genetic code for a protein is of fundamental importance in understanding the characteristics of the protein concerned.

The process used to determine the sequence of the bases in a DNA molecule is referred to as DNA sequencing. Among the techniques of DNA sequencing, the enzymatic method developed by Sanger et al. (1) is most popular. It is based on the ability of a DNA polymerase to extend a primer annealed to the DNA template to be sequenced in the presence of four normal deoxynucleotide triphosphates (dNTPs), namely, dATP, dCTP, dGTP and dTTP, and on the ability of the nucleotide analogs, the dideoxynucleotide triphosphates (ddNTPs), namely, ddATP, ddCTP, ddGTP and ddTTP, to terminate the extension of the elongating deoxynucleotide polymers at various lengths.

In enzymatic polymerization reactions using double-stranded DNA templates, it is necessary to denature the target DNA fragments. To that end, heating a double-stranded DNA, usually to 95° C., denatures the molecule to create two complementary single-stranded DNA fragments. In an enzymatic DNA polymerization reaction, after a primer annealed to its complementary sequence on a single-stranded template has been extended to form a new DNA strand, the latter can be separated from its template when heated to 95° C., which is above its melting temperature. The single-stranded template is again available for annealing with an oligonucleotide primer upon cooling, ready for another cycle of enzymatic DNA synthesis in the presence of a functioning DNA polymerase and dNTPs. Usually a heat-resistant DNA polymerase, which can survive the heating to 95° C. and is active at temperature between 55 and 72° C., is employed in the system so that no fresh enzyme needs to be added to initiate each cycle of DNA synthesis after denaturing at high temperature. When a primer is mixed in excess with a template and the temperature cycles repeat for a plurality of times, the number of the extended single-stranded target fragments increases one fold per cycle. When a set of ddNTPs, including all four A, C, G and T bases, or their analogs, is present as chain terminators in the reaction system, numerous single-stranded DNA fragments of various lengths, all having the same primer at their 5′end and terminating with a specific ddNTP or its analog, which may be labeled with a fluorescent dye as a reporter, at the 3′end are generated. This forms the basis of automated DNA cycle sequencing with fluorescent dye-labeled DNA terminators.

When a set of two primers (one forward and one reverse) complementary to the two ends of a target sequence on a double-stranded DNA template is used in the above-described DNA cycle sequencing system, the newly extended DNA strands can serve as additional templates in the subsequent cycle of DNA synthesis. Hence the copy number of the target sequence fragments is amplified exponentially if the heating cycle is repeated for plurality of times. This forms the basis of the Polymerase Chain Reaction (PCR).

In practice, both automated cycle sequencing with fluorescent dye-labeled terminators and PCR have depended on the use of heat-resistant DNA polymerases, such as Thermus aquaticus DNA polymerase (Taq) and its equivalents, which can survive the heating temperature of 95° C. However, heat-resistant polymerases are usually associated with low processivity, and may lose their sequence-specific polymerase activity under certain unpredictable conditions, especially when GC-rich DNA segments (that is, segments containing a significantly higher content of guanine and cytosine, relative to the content of thymine and adenine) in a template are to be amplified or to be sequenced. Therefore, attempts have been made to develop conditions suitable for low temperature cycle sequencing and for low temperature cycle PCR using thermolabile DNA polymerases, which, in general, have higher fidelity and higher processivity than the heat-resistant DNA polymerases. For example, in U.S. Pat. No. 5,432,065, there is described the use of glycerol or ethylene glycol—at a final concentration of 40% (v/v)—to lower the melting temperature of the template DNA and to extend the primer at temperatures below 80° C. in a cycle primer extension reaction, in conjunction with a DNA polymerase from Bacillus caldotenax and from a Klenow fragment. However, it was later found that even at such a high concentration of glycerol, the Klenow DNA polymerase was not useful for low temperature cycle primer extension. Lowering the glycerol concentration to 17% (v/v) in the reaction mixture with the addition of proline appeared to protect the Klenow polymerase activity in cycle PCR at the temperature range between 70° C. and 37° C. (Iakobashvili and Lapidot) Significantly, neither of these procedures for low temperature cycle primer extension has been shown to generate high quality sequence-specific PCR products, or has been shown to generate reaction products suitable for DNA sequencing. In the Iakobashvili and Lapidot report, the PCR products generated in low-temperature cycle primer extension have not proved to be sequence-specific, especially when primers of 20-25 base pairs (bp) in length were used. Although the application was said to be successful for cycle extension of long primers (such as 30-35 bp in length) using the Klenow polymerase at the low temperature range, the system has not been shown to generate useful sequence-specific amplification products from such long primers. Since most primers used for DNA cycle sequencing and for PCR are shorter than 30 bp in length, there is a need for a low-temperature cycling system with which sequence-specific extension of primers of shorter than 30 bp (preferably about 20 bp) can be achieved to generate useful amplification DNA products for sequencing and for further molecular analysis.

### SUMMARY OF THE INVENTION

The invention described in this application has provided such a system. With the above issues in mind, the inventors have developed methods for extending a primer or a pair of primers in cycle DNA amplification for automated cycle sequencing and PCR. In particular, the methods contemplate moderately thermostable DNA polymerases in the presence of a low concentration of glycerol or ethylene glycol, or the mixtures thereof, as an agent to reduce the melting temperature of DNA (that is, the temperature at which the double-strands of DNA are denatured). The inventors observed that at a certain concentration range, glycerol and/or ethylene glycol not only reduced the melting temperature of the DNA template, but also increased the polymerization activity of the moderately thermostable DNA polymerases. In these enzymatic reaction systems, the temperature range of cycling is between 70° C. and 37° C.—much lower than what is usually required for denaturing DNA. In addition, the methods use highly processive, moderately thermostable DNA polymerases preferably derived from Bacillus stearothermophilus, Bacillus caldotenax or Bacillus caldolyticus. These polymerases have an optimum reaction temperature at 65° C., but are rapidly inactivated above 70° C.; thus, they are quite useful as the polymerizing enzymes for the cycle primer extension to overcome some of the shortcomings of the heat-resistant DNA polymerases, such as Taq and its corresponding mutants, and of the heat-labile DNA polymerases, such as the Klenow fragment. The moderately thermostable DNA polymerases may be in their natural state (e.g., purified from the organisms), or modified.

Thus, in a broad embodiment, the invention contemplates a method for extending a primer (or a pair of primers) using an enzymatic cycle primer extension reaction at low cycling temperatures (that is, temperatures below about 80° C.), in a reaction mixture composition comprising between about 10% and about 20% (and preferably about 15%) (v/v) glycerol, ethylene glycol, or a mixture thereof, in the presence of a moderately thermostable (also referred to as mesophilic) DNA polymerase. (By “enzymatic cycle primer extension reaction”, it is meant that in excess of primer over template, the limited number of template molecules can be used repeatedly for DNA polymerization catalyzed by a functional DNA polymerase when the temperature of the reaction mixture fluctuates repeatedly between the levels required for denaturing, annealing and primer extension in cycles. By “moderately thermostable DNA polymerase, it is meant polymerases that have an optimum reaction temperature at 65° C., and which are rapidly inactivated above 70° C.) For instance, DNA template may be mixed with a primer (or a pair of primers) and a natural or a modified form of a moderately thermostable DNA polymerase from one of Bacillus stearothermophilus, Bacillus caldotenax or Bacillus caldolyticus, in a solution containing between about 10% and about 20% (v/v) (preferably about 15% (v/v)) glycerol, ethylene glycol, or a mixture thereof. The reaction may be carried out under conditions that the cycle reaction temperature fluctuates between a melting temperature of about 70° C. and a cooling (or annealing) temperature of about 37° C., so that the DNA polymerase repeatedly extends the primer or pair of primers at the temperature between about 45° C. and 50° C. The method may include the further step of repeating the cycle primer extension reaction, as many times as is desired.

In another embodiment, copies of a selected segment of a double-stranded DNA are amplified in the presence of a forward primer and a reverse primer (where both may be of various lengths) to the template by repeated heating and cooling (or annealing) cycles (such as, for instance, in a PCR). Here, again, the reaction is run at low temperatures (that is, temperatures below about 80° C.), in a reaction mixture composition comprising between about 10% and about 20% (and preferably about 15%) (v/v) glycerol, ethylene glycol, or a mixture thereof, in the presence of one of the moderately thermostable DNA polymerases described above. The reaction may be carried out under conditions that the reaction temperature fluctuates between a melting temperature of about 70° C. and a cooling (or annealing) temperature of about 37° C., so that the DNA polymerase repeatedly extends the forward and reverse primers at the temperature of between about 45° C. and 50° C. The method may include the further step of repeating the reaction, as many times (e.g., cycles) as is desired.

In a preferred embodiment, the DNA polymerase is one of those described in U.S. Pat. Nos. 5,747,298, 5,834,253 or 6,165,765. Preferably the DNA polymerase has an amino acid sequence that shares not less than 95% homology of a DNA polymerase isolated from Bacillus stearothermophilus, Bacillus caldotenax or Bacillus caldolyticus.

In a further embodiment, molecules of a single primer of various lengths are extended with specific nucleotide terminations in the presence of ddNTPs or their analogs for cycle sequencing.

The invention also contemplates a method for extending the molecules of a single primer annealed to a single-stranded copy of the doubled-stranded DNA product amplified in vitro without prior isolation or purification for direct cycle sequencing. For instance, a diluted crude amplified reaction product (preferably generated with a low-temperature PCR reaction catalyzed by a moderately thermostable DNA polymerase as described herein) is used as a template and mixed with an excess amount of a sequencing primer, the four standard ddNTP terminators (ddATP, ddGTP, ddTTP and ddCTP) fluorescently labeled (or their corresponding analogs fluorescently labeled), a moderately thermostable DNA polymerase (preferably one with a reduced innate selective discrimination against incorporation of a subset of dye-labeled ddNTPs), a suitable concentration of dNTPs (dATP, dGTP, dTTP and dCTP), and a composition comprising a buffer in a solution containing between about 10% and about 20% (preferably 15%) (v/v) of glycerol, ethylene glycol, or mixture thereof. A standard cycle primer extension reaction(s) may then be run at a temperature below 80° C. for a sufficient number of times to extend the sequencing primer molecules to desired varying lengths, which extended molecules will be terminated specifically by fluorescently labeled ddNTPs or their corresponding analogs. Preferably, the cycle reaction temperature fluctuates between a melting temperature of about 70° C. and a cooling/annealing temperature of about 37° C.

In one preferred embodiment, the method of sequencing a DNA strand may comprise the steps of:

i) hybridizing a primer to a DNA template to be sequenced; and

ii) extending the primer using one of the above-described DNA polymerases, in the presence of a solution containing between about 10% and about 20% (v/v) (preferably about 15% (v/v)) glycerol, ethylene glycol, or a mixture thereof, adequate amounts of the deoxynucleotide bases dATP, dGTP, dCTP and dTTP, and the four dideoxynucleotide terminators or their analogs, whereby the cycle reaction temperature fluctuates between a melting temperature of about 70° C. and a cooling or annealing temperature of about 37° C., and under such conditions that the DNA strand is sequenced. Preferably one of the deoxynucleotides is radioisotope-labeled, or the primer molecules are fluorescent dye-labeled, and more preferably all dideoxynucleotide terminators are fluorescent dye-labeled.

In another embodiment, the invention entails a dry or liquid ready-to-use reaction mixture or kit suitable for use in a low-temperature cycle primer extension reaction at temperatures below about 80° C. This reaction mixture or kit comprises a moderately thermostable DNA polymerase (such as one of those described above) that is pre-mixed with at least one enzymatic DNA primer extension reaction component suitable for use in DNA amplification or for specific extension terminations with dideoxyribonucleotide analogs. The reaction mixture is preferably pre-distributed into microcentrifuge tubes or in multiple-well plates, such as, for instance, those that are suitable for large-scale automated PCR or for large-scale automated DNA sequencing. This ready-to-use reaction mixture or kit can be stored at room temperature between about 22° C. and about 25° C. for at least eight weeks without losing its specific polymerization activity for DNA primer amplification or extension terminations.

Further objects and advantages of the invention will become apparent from the description and examples below.

### BRIEF DESCRIPTION OF THE DRAWINGS

FIG. 1 is a graph illustrating the effect of glycerol on 5′-3′ polymerization activity on Bst-II DNA polymerase.

FIG. 2 is a picture of an electrophoresis gel (1% agarose), showing the results of low-temperature amplification with a moderately thermostable DNA polymerase in 40% glycerol.

FIG. 3A is a picture of an electrophoresis gel (1% agarose), showing the results of low-temperature cycle primer extension in 35% glycerol (lane 1) and in 15% glycerol (lane 2) with amplified products having a length of 250 base pairs. FIG. 3B is a picture of an electrophoresis gel (1% agarose), showing the results of low-temperature cycle primer extension in 35% glycerol (lane 1) and in 15% glycerol (lane 2) with amplified products having a length of 400 base pairs. FIG. 3C is a picture of an electrophoresis gel (1% agarose), showing the results of low-temperature cycle primer extension in 35% glycerol (lane 1) and in 15% glycerol (lane 2) with amplified products having a length of 1 kilobase. FIG. 3D is a picture of an electrophoresis gel (1% agarose), showing the results of low-temperature cycle primer extension in 35% glycerol (lane 1) and in 15% glycerol (lane 2) with amplified products having a length of 2 kilobases.

FIG. 4 is a picture of an electrophoresis gel (1% agarose), showing the results of low-temperature cycle extension reaction of 17 mer and 30 mer primers with moderately thermostable DNA polymerases and Klenow fragment. The reaction products with 17 mer primers are A1 (Klenow fragment using the Iakobashvili and Lapidot system), A2 (Klenow fragment with the Bst system), A3 (Bst-I polymerase with the Bst system), A4 (Bst-II polymerase with the Bst system), and A5 (Bca polymerase with the Bst system). The reaction products with 30 mer primers are B1 (Klenow fragment using the Iakobashvili and Lapidot system), B2 (Klenow fragment with the Bst system), B3 (Bst-I polymerase with the Bst system), B4 (Bst-II polymerase with the Bst system), and B5 (Bca polymerase with the Bst system).

FIGS. 5A and 5B represent two automated fluorescent DNA sequencing tracings of a GC-rich segment, comparing the performance of AmpliTaq™ in the ABI Prism™ BigDye™ Terminator cycle sequencing kit (5A) with that of the Bst-II cycle sequencing system (5B).

FIG. 6 is a picture of an electrophoresis gel (1% agarose), showing the results of cycle primer extension reactions conducted at various temperature steps, using a moderately thermostable DNA polymerase (Bst-II), with no glycerol and with 15% glycerol.

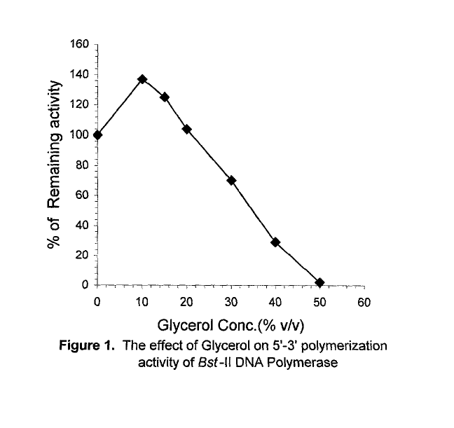

Additional details about FIGS. 1-6 are included in the description and examples that follow.

### DETAILED DESCRIPTION OF THE INVENTION

As noted above, this invention entails a unique combination of a moderately thermostable DNA polymerase (such as Bacillus stearothermophilus, Bacillus caldotenax or Bacillus caldolyticus) in the presence of a low concentration of an agent selected from the group consisting of glycerol, ethylene glycol and mixtures of these, to provide a way to extend a primer (or pair of primers) in cycle DNA amplification for automated cycle sequencing and PCR at temperatures below about 80° C. The inventors discovered that both glycerol and ethylene glycol at low concentrations increase the sequence-specific DNA polymerization activity of the moderately thermostable DNA polymerases in vitro. At higher concentrations, for example greater than 35%, both glycerol and ethylene glycol exhibit a detrimental inhibitory effect on the DNA polymerization activity of these enzymes. However, the inventors achieved a reaction mixture with an optimum concentration of glycerol or ethylene glycol, in which double-stranded DNA templates are denatured at 70° C. while the polymerization activity of the moderately thermostable DNA polymerases can be preserved during low temperature sequence-specific cycle primer extension.

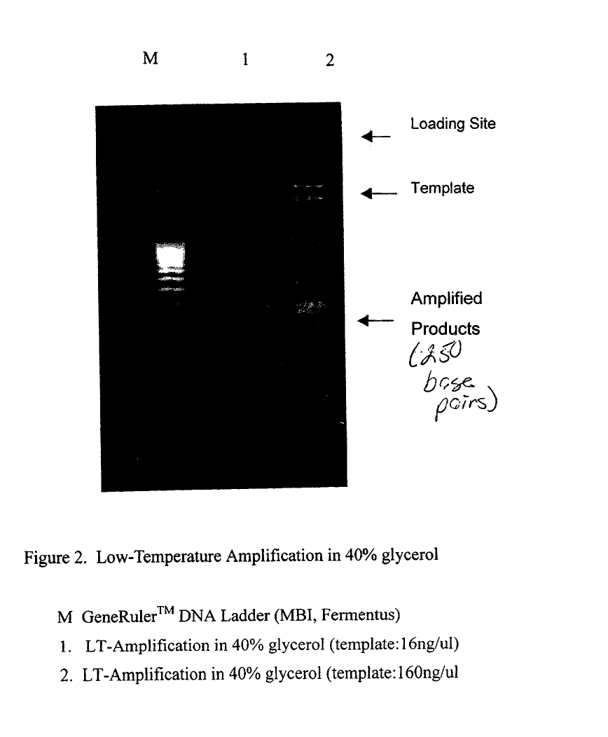

For instance, the inventors first observed that at the optimum enzymatic reaction temperature of 65° C., a final concentration of glycerol of up to about 20% increased the 5′-3′ polymerization activity of the moderately thermostable DNA polymerases (for instance, see FIG. 1). However, when the concentrations of glycerol was increased to greater than about 35% it invariably suppressed this enzymatic activity. When the glycerol concentration increases to 40% (v/v), this group of DNA polymerases usually lost more than two thirds (⅔) of the original polymerization activity. It was found that low-temperature cycle extensions with moderately thermostable DNA polymerases in a solution containing 40% of glycerol generates poorly defined non-specific amplified products of varying fragment sizes (for instance, see FIG. 2).

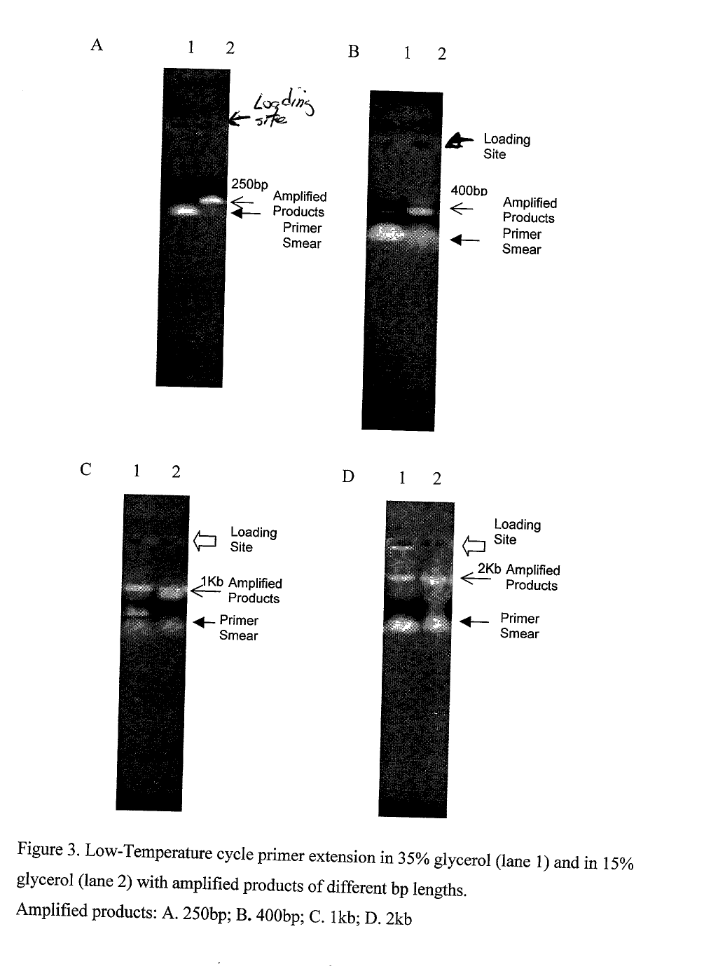

The inventors found that in the presence of 35% glycerol, low temperature cycle primer extension reactions catalyzed by moderately thermostable DNA polymerases, such as a Bst mutant or Bca, resulted in no amplification at all for a target product of 250 bp and 400 bp long. When longer segments of DNA—for example, 1 kb and 2 kb in length—are the amplification target, there is evidence of amplification; but the reaction products are non-specific. (For instance, see FIG. 3).

It was further found that with a low concentration of glycerol, for example 15% (v/v), and a Bst mutant or Bca DNA polymerase, sequence-specific amplification products of less than 250 bp to more than 2 kb in length can be generated (see, for example, FIG. 3).

Thus, a low concentration of glycerol or ethylene glycol, for example between about 10% and about 20% v/v, preferably 15%, can be used to lower the DNA melting temperature for cycle primer extension in conjunction with a moderately thermostable DNA polymerase to generate sequence-specific amplification products.

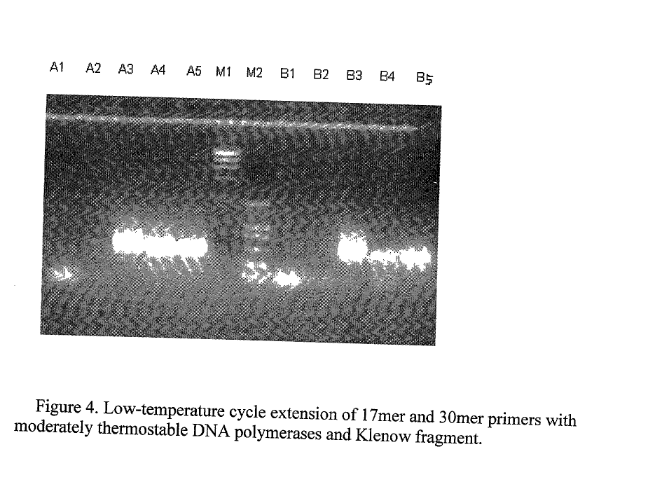

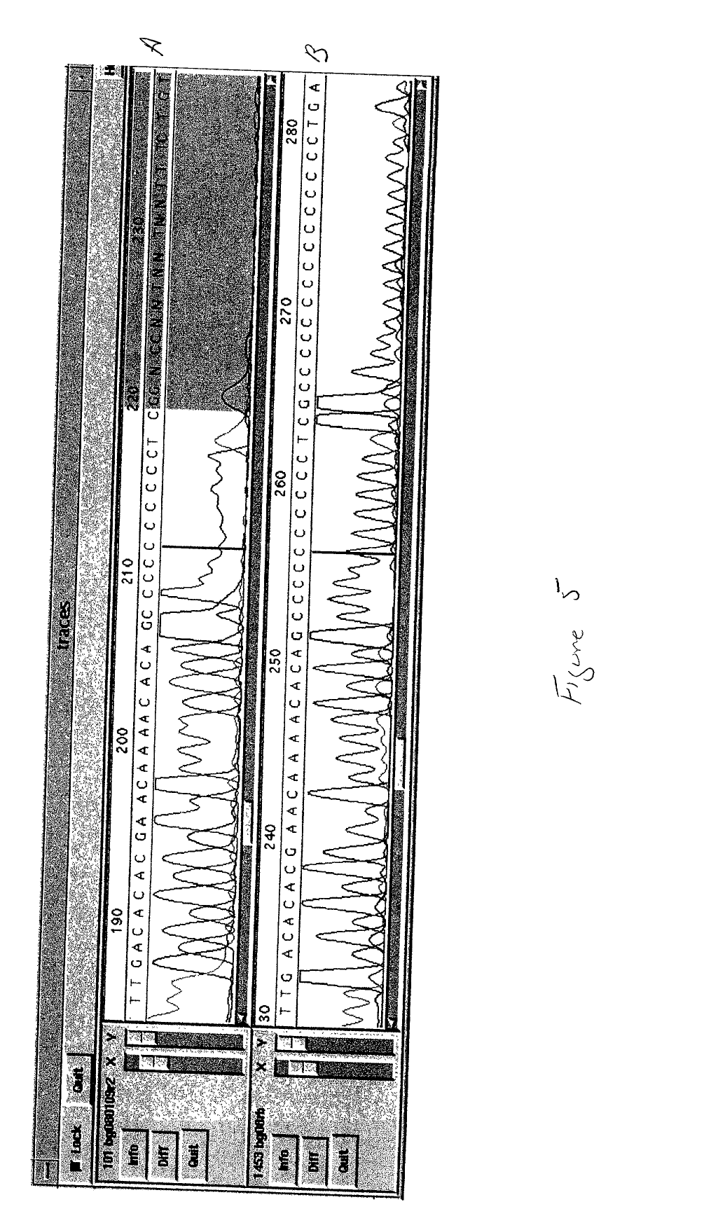

In this low-temperature cycle extension system, DNA fragments of a wide range in length, including those having less than 30 base pairs, even shorter than 20 base pairs in length can be used as the primers for sequence-specific extensions (for instance, see FIG. 4). The thermolabile DNA polymerases, such as the Klenow fragment, fail to generate any significant amount of amplification products useful for further analysis (see, for instance, FIG. 4). ThermoSequenase™ and AmpliTaq™, both being modified forms of the heat-resistant Taq DNA polymerase, cannot generate sequence-specific products useful for further analysis in the low-temperature cycle extension system. However, under their optimum high-temperature (melting at 95° C.) cycle extension conditions, these two enzymes may extend the primers annealed to most DNA templates until a GC-rich segment is encountered. Compared with the results of using a moderately thermostable DNA polymerase for low-temperature cycle extension, the low processivity of the heat-resistant DNA polymerases under high temperature cycle extension becomes evident when they are used for automated cycle sequencing of known GC-rich templates. The heat-resistant DNA polymerases generate no sequence-specific ddNTP terminations down-stream to the GC-rich segment of the template whereas a moderately thermostable enzyme, for example the mutated Bst-II, can successfully overcome the GC-rich obstacle during DNA polymerase cycle extensions (see, for instance, FIG. 5).

As indicated above, the use of moderately thermostable DNA polymerases is quite critical to the methods of this invention. By “moderately thermostable” it is meant that these polymerases have an optimum reaction temperature at 65° C., but are rapidly inactivated above 70° C. To that end, the invention contemplates DNA polymerases obtained or derived from one or more of Bacillus stearothermophilus (Bst), Bacillus caldotenax (Bca) or Bacillus caldolyticus (Bcy). All three of these organisms are classified as mesophilic microbes because, although their DNA polymerases are referred to as thermostable (most active at 65° C.), they are inactivated at 70° C. or above. This is contrasted with other enzymes, such as Taq, which are truly thermophilic—that is, the Taq DNA polymerase tolerates and remains active at temperatures higher than 95° C. These mesophilic bacillus strains, especially Bacillus stearothermophilus, produce DNA polymerases that are useful in DNA cycle sequencing and PCR applications.

In a preferred embodiment, a moderately thermostable (also sometimes referred to as mesophilic) DNA polymerase may have proofreading 3′-5′ exonuclease activity during DNA primer extension over a template, such that the DNA polymerase functions to excise mismatched nucleotides from the 3′ terminus of the DNA strand at a faster rate than the rate at which the DNA polymerase functions to remove nucleotides matched correctly with nucleotides of the template. Such DNA polymerases are also described by the inventors in U.S. Pat. Nos. 5,834,253, 5,747,298, and 6,165,765 (the contents of all of which are incorporated herein by reference in their entirety). One strain of Bacillus stearothermophilus (designated strain No. 320 for identification purposes; described in U.S. Pat. No. 5,747,298) produces a DNA polymerase (designated Bst 320) with a proof-reading 3′-5′ exonuclease activity which is absent in DNA polymerases isolated from other strains of Bacillus stearothermophilus. (For this invention, the term “proof-reading” is intended to denote that the DNA polymerase is capable of removing mismatched nucleotides from the 3′ terminus of a newly formed DNA strand at a faster rate than the rate at which nucleotides correctly matched with the nucleotides of the template are removed during DNA sequencing.) The strain Bst 320 was deposited on Oct. 30, 1995 in the American Type Culture Collection, located at 12301 Parklawn Drive, Rockville, Md. 20852, and has been given ATCC Designation No. 55719. The DNA polymerase isolated from Bst 320 is composed of 587 amino acids as are the DNA polymerases of other known strains of Bacillus stearothermophilus, such as, for instance, the strains deposited by Riggs et al (Genbank Accession No. L42111) and by Phang et al. (Genbank Accession No. U23149). However, the Bst 320 shares only 89.1% sequence identity at protein level with the Bacillus stearothermophilus DNA polymerase deposited by Riggs et al., and shares only 87.4% sequence identity at protein level with the Bacillus stearothermophilus DNA polymerase deposited by Phang et al. For comparison, the above-referenced enzyme deposited by Riggs et al. and the enzyme deposited by Phang et al. share 96.9% of their amino acid sequence identity.

The inventors studied a thermostable DNA polymerase isolated from a different species, Bacillus caldotenax (Bca), which also has an optimum active temperature at 65° C. The inventors discovered that the Bst 320 DNA polymerase shares 88.4% of the amino acid sequence identity with Bca DNA polymerase (Uemori et al. J. Biochem. 113: 401-410, 1993). Based on homology of the amino acid sequences, Bst 320 DNA polymerase is as close to DNA polymerases isolated from Bacillus stearothermophilus as to the DNA polymerase isolated from Bacillus caldotenax, i.e. another species of bacillus. It was also discovered that both Bst 320 DNA polymerase and Bca DNA polymerase functionally exhibit 3′-5′ exonuclease activity, which is not associated with known amino acid sequence exonuclease motifs I, II and III as in the E. coli DNA polymerase I model, or other known Bacillus stearothermophilus polymerases.

One preferred Bst DNA polymerase is isolated from strain 320 with an amino acid sequence as follows:

Amino acid sequence:

(SEQ ID NO:2)
AEGEKPLEEM EFAIVDVITE EMLADKAALV VEVMEENYHD
APIVGIALVNE HGRFFMRPE TALADSQFLA WLADETKKKS
MFDAKRAVVA LKWKGIELRG VAFDLLLAAY LLNPAQDAGD
IAAVAKMKQY EAVRSDEAVY GKGVKRSLPD EQTLAEHLVR
KAAAIWALEQ PFMDDLRNNE QDQLLTKLEH ALAAILAEME
FTGVNVDTKR LEQMGSELAE QLRAIEQRIY ELAGQEFNIN
SPKQLGVILF EKLQLPVLKK TKTGYSTSAD VLEKLAPHHE
IVENILHYRQ LGKLQSTYIE GLLKVVRPDT KVHTMFNQA
LTQTGRLSSA EPNLQNIPIR LEEGRKIRQA FVPSEPDWLI
FAADYSQIEL RVLAHIADDD NLIEAFQRDL DIHTKTAMDI
FQLSEEEVTA NMRRQAKAV NFGIVYGISDY GLAQNLNITR
KEAAEFIERY FASFPGVKQY MENIVQEAKQ KGYVTTLLHR
RRYLPDITSR NFNVRSFAER TAMNTPIQGS AADIIKKAMI
DLAARLKEEQ LQARLLLQVH DELILEAPKE EIERLCELVP
EVMEQAVTLR VPLKVDYHYG PTWYDAK

The characters represent the following amino acids:

where,

A: alanine (Ala)

C: cysteine (Cys)

D: aspartic acid (Asp)

E: glutamic acid (Glu)

F: phenylanaline (Phe)

G: glycine (Gly)

H: histidine (His)

I: isoleucine (Ile)

K: lysine (Lys)

L: leucine (Leu)

M: methionine (Met)

N: asparagine (Asn)

P: proline (Pro)

Q: glutamine (Gln)

R: arginine (Arg)

S: serine (Ser)

T: threonine (Thr)

V: valine (Val)

W: trytophan (Trp)

Y: tyrosine (Tyr)

This Bst 320 DNA polymerase is characterized by possessing a proofreading 3′-5′ exonuclease activity.

The nucleotide sequence encoding the Bst 320 DNA polymerase is indicated in SEQ ID NO:1, below.

DNA sequence (isolated/purified):

GCCGAAGGGG AGAAACCGCT TGAGGAGATG GAGTTTGCCA
TCGTTGACGT CATTACCGAA GAGATGCTTG CCGACAAGGC
AGCGCTTGTC GTTGAGGTGA TGGAAGAAAA CTACCACGAT
GCCCCGATTG TCGGAATCGC ACTAGTGAAC GAGCATGGGC
GATTTTTTAT GCGCCCGGAG ACCGCGCTGG CTGATTCGCA
ATTTTTAGCA TGGCTTGCCG ATGAAACGAA GAAAAAAAGC
ATGTTTGACG CCAAGCGGGC AGTCGTTGCC TTAAAGTGGA
AAGGAATTGA GCTTCGCGGC GTCGCCTTTG ATTTATTGCT
CGCTGCCTAT TTGCTCAATC CGGCTCAAGA TGCCGGCGAT
ATCGCTGCGG TGGCGAAAAT GAAACAATAT GAAGCGGTGC
GGTCGGATGA AGCGGTCTAT GGCAAAGGCG TCAAGCGGTC
GCTGCCGGAC GAACAGACGC TTGCTGAGCA TCTCGTTCGC
AAAGCGGCAG CCATTTGGGC GCTTGAGCAG CCGTTTATGG
ACGATTTGCG GAACAACGAA CAAGATCAAT TATTAACGAA
GCTTGAGCAC GCGCTGGCGG CGATTTTGGC TGAAATGGAA
TTCACTGGGG TGAACGTGGA TACAAAGCGG CTTGAACAGA
TGGGTTCGGA GCTCGCCGAA CAACTGCGTG CCATCGAGCA
GCGCATTTAC GAGCTAGCCG GCCAAGAGTT CAACATTAAC
TCACCAAAAC AGCTCGGAGT CATTTTATTT GAAAAGCTGC
AGCTACCGGT GCTGAAGAAG ACGAAAACAG GCTATTCGAC
TTCGGCTGAT GTGCTTGAGA AGCTTGCGCC GCATCATGAA
ATCGTCGAAA ACATTTTGCA TTACCGCCAG CTTGGCAAAC
TGCAATCAAC GTATATTGAA GGATTGTTGA AAGTTGTGCG
CCCTGATACC GGCAAAGTGC ATACGATGTT CAACCAAGCG
CTGACGCAAA CTGGGCGGCT CAGCTCGGCC GAGCCGAACT
TGCAAAACAT TCCGATTCGG CTCGAAGAGG GGCGGAAAAT
CCGCCAAGCG TTCGTCCCGT CAGAGCCGGA CTGGCTCATT
TTCGCCGCCG ATTACTCACA AATTGAATTG CGCGTCCTCG
CCCATATCGC CGATGACGAC AATCTAATTG AAGCGTTCCA
ACGCGATTTG GATATTCACA CAAAAACGGC GATGGACATT
TTCCAGTTGA GCGAAGAGGA AGTCACGGCC AACATGCGCC
GCCAGGCAAA GGCCGTTAAC TTCGGTATCG TTTACGGAAT
TAGCGATTAC GGATTGGCGC AAAACTTGAA CATTACGCGC
AAAGAAGCTG CCGAATTTAT CGAACGTTAC TTCGCCAGCT
TTCCGGGCGT AAAGCAGTAT ATGGAAAACA TAGTGCAAGA
AGCGAAACAG AAAGGATATG TGACAACGCT GTTGCATCGG
CGCCGCTATT TGCCTGATAT TACAAGCCGC AATTTCAACG
TCCGCAGTTT TGCAGAGCGG ACGGCCATGA ACACGCCAAT
TCAAGGAAGC GCCGCTGACA TTATTAAAAA AGCGATGATT
GATTTAGCGG CACGGCTGAA AGAAGAGCAG CTTCAGGCTC
GTCTTTTGCT GCAAGTGCAT GACGAGCTCA TTTTGGAAGC
GCCAAAAGAG GAAATTGAGC GATTATGTGA GCTTGTTCCG
GAAGTGATGG AGCAGGCCGT TACGCTCCGC GTGCCGCTGA
AAGTCGACTA CCATTACGGC CCAACATGGT ATGATGCCAA
ATAA (1764 nucleotides total)

The characters represent the following nucleotides:

A: Adenosine T: Thymidine C: Cytidine G: Guanosine

However, while quite useful with this invention, a disadvantage of the DNA polymerases of the mesophilic strains Bacillus stearothermophilus, Bacillus caldotenax or Bacillus caldolyticus, is that during DNA sequencing they all exhibit a high degree of selective discrimination against incorporation of certain particular members of fluorescent dye-labeled ddNTPs, namely the fluorescent dye-labeled ddCTP and fluorescent dye-labeled ddATP, as terminators onto the 3′ end of the extending DNA fragments during enzymatic reaction. This peculiar characteristic of selective discrimination against incorporation of fluorescent dye-labeled ddCTP and ddATP of the natural DNA polymerases isolated from Bacillus stearothermophilus and Bacillus caldotenax was only recognized recently by the inventors. Such selective discrimination is apparently sequence-related, and cannot be corrected or compensated by mere adjustment of the concentrations of the dNTPs.

Therefore, in another preferred embodiment the DNA polymerase used is a mesophilic bacillus DNA polymerase (such as Bacillus stearothermophilus, Bacillus caldotenax and Bacillus caldolyticus) which, during dye-labeled terminator automated DNA cycle sequencing, reduces the innate selective discrimination against the incorporation of fluorescent dye-labeled ddCTP and fluorescent dye-labeled ddATP, without increasing the rate of incorporation of the other two dye-labeled ddNTP terminators (ddTTP and ddGTP) excessively. Such DNA polymerases are described by the inventors in U.S. Pat. No. 6,165,765 (the contents of all of which are incorporated herein by reference in their entirety).

For example, polymerases having this ability to reduce selective discrimination may be obtained or otherwise derived from a strain of Bacillus stearothermophilus, Bacillus caldotenax and Bacillus caldolyticus, or made synthetically, where the amino acid sequences of the naturally-occurring DNA polymerase have leucine-glutamate-glutamate at positions corresponding respectively to positions 342-344 of Bst 320 DNA polymerase and phenylalanine at a position corresponding to position 422 of Bst 320 DNA polymerase. For instance, DNA polymerases derived from other strains of Bacillus stearothermophilus, Bacillus caldotenax and Bacillus caldolyticus, may be easily modified using conventional DNA modification techniques to include the amino acid or nucleotide substitutions identified above.

The following amino acid sequence represents the modified Bst 320 DNA polymerase (also referred to herein as “Bst II” or “HiFi Bst II”) as another preferred embodiment of this invention, modified from the naturally-occurring Bst 320 DNA polymerase at positions 342-344 to substitute threonine, proline and leucine, respectively, for leucine, glutamate and glutamate, and at position 422 to substitute tyrosine for phenylalanine.

Amino acid sequence (SEQ ID:No 4):

Amino acid sequence:

(SEQ ID:No 4)
MAEGEKPLEEMEFAIVDVITEEMLADKAALVVEVMEENYHDAPIVGIALV
NEHGRFFMRPETALADSQFLAWLADETKKKSMFDAKRAVVALKWKGIELR
GVAFDLLLAAYLLNPAQDAGDIAAVAKMKQYEAVRSDEAVYGKGVKRSLP
DEQTLAEHLVRKAAAIWALEQPFMDDLRNNEQDQLLTKLEHALAAILAEM
EFTGVNVDTKRLEQMGSELAEQLRAIEQRIYELAGQEFNINSPKQLGVIL
FEKLQLPVLKKTKTGYSTSADVLEKLAPHHEIVENILHYRQLGKLQSTYI
EGLLKVVRPDTGKVHTMFNQALTQTGRLSSAEPNLQNIPIRTPLGRK
IRQAFVPSEPDWLIFAADYSQIELRVLAHIADDDNLIEAFQRDLDIHTKT
AMDIFQLSEEEVTANMRRQAKAVNYGIVYGISDYGLAQNLNITRKEA
AEFIERYFASFPGVKQYMENIVQEAKQKGYVTTLLHRRRYLPDITSRNFN
VRSFAERTAMNTPIQGSAADIIKKAMIDLAARLKEEQLQARLLLQVHDEL
ILEAPKEEIERLCELVPEVMEQAVTLRVPLKVDYHYGPTWYDAK

The underlined amino acids are substituted amino acids produced by site-directed mutation of the naturally-occurring Bst 320 DNA polymerase.

The modified Bst 320 DNA polymerase is encoded by a DNA sequence such as the following (SEQ ID NO:3):

(SEQ ID NO:3)
ATG GCCGAAGGGG AGAAACCGCT TGAGGAGATG
GAGTTTGCCA TCGTTGACGT CATTACCGAA GAGATGCTTG
CCGACAAGGCAGCGCTTGTC GTTGAGGTGA TGGAAGAAAA
CTACCACGATGCCCCGATTG TCGGAATCGC ACTAGTGAAC
GAGCATGGGCGATTTTTTAT GCGCCCGGAG ACCGCGCTGG
CTGATTCGCAATTTTTAGCA TGGCTTGCCG ATGAAACGAA
GAAAAAAAGCATGTTTGACG CCAAGCGGGC AGTCGTTGCC
TTAAAGTGGAAAGGAATTGA GCTTCGCGGC GTCGCCTTTG
ATTTATTGCTCGCTGCCTAT TTGCTCAATC CGGCTCAAGA
TGCCGGCGATATCGCTGCGG TGGCGAAAAT GAAACAATAT
GAAGCGGTGCGGTCGGATGA AGCGGTCTAT GGCAAAGGCG
TCAAGCGGTCGCTGCCGGAC GAACAGACGC TTGCTGAGCA
TCTCGTTCGCAAAGCGGCAG CCATTTGGGC GCTTGAGCAG
CCGTTTATGGACGATTTGCG GAACAACGAA CAAGATCAAT
TATTAACGAAGCTTGAGCAC GCGCTGGCGG CGATTTTGGC
TGAAATGGAATTCACTGGGG TGAACGTGGA TACAAAGCGG
CTTGAACAGATGGGTTCGGA GCTCGCCGAA CAACTGCGTG
CCATCGAGCAGCGCATTTAC GAGCTAGCCG GCCAAGAGTT
CAACATTAACTCACCAAAAC AGCTCGGAGT CATTTTATTT
GAAAAGCTGCAGCTACCGGT GCTGAAGAAG ACGAAAACAG
GCTATTCGACTTCGGCTGAT GTGCTTGAGA AGCTTGCGCC
GCATCATGAAATCGTCGAAA ACATTTTGCA TTACCGCCAG
CTTGGCAAACTGCAATCAAC GTATATTGAA GGATTGTTGA
AAGTTGTGCGCCCTGATACC GGCAAAGTGC ATACGATGTT
CAACCAAGCGCTGACGCAAA CTGGGCGGCT CAGCTCGGCC
GAGCCGAACTTGCAAAACAT TCCGATTCGG ACCCCACTGG
GGCGGAAAATCCGCCAAGCG TTCGTCCCGT CAGAGCCGGA
CTGGCTCATT TTCGCCGCCG ATTACTCACA AATTGAATTG
CGCGTCCTCGCCCATATCGC CGATGACGAC AATCTAATTG
AAGCGTTCCAACGCGATTTG GATATICACA CAAAAACGGC
GATGGACATTTTCCAGTTGA GCGAAGAGGA AGTCACGGCC
AACATGCGCCGCCAGGCAAA GGCCGTTAAC TACGGTATCG
TTTACGGAATTAGCGATTAC GGATTGGCGC AAAACTTGAA
CATTACGCGCAAAGAAGCTG CCGAATTTAT CGAACGTTAC
TTCGCCAGCTTTCCGGGCGT AAAGCAGTAT ATGGAAAACA
TAGTGCAAGAAGCGAAACAG AAAGGATATG TGACAACGCT
GTTGCATCGGCGCCGCTATT TGCCTGATAT TACAAGCCGC
AATTTCAACGTCCGCAGTTT TGCAGAGCGG ACGGCCATGA
ACACGCCAATTCAAGGAAGC GCCGCTGACA TTATTAAAAA
AGCGATGATTGATTTAGCGG CACGGCTGAA AGAAGAGCAG
CTTCAGGCTCGTCTTTTGCT GCAAGTGCAT GACGAGCTCA
TTTTGGAAGCGCCAAAAGAG GAAATTGAGC GATTATGTGA
GCTTGTTCCGGAAGTGATGG AGCAGGCCGT TACGCTCCGC
GTGCCGCTGAAAGTCGACTA CCATTACGGC CCAACATGGT
ATGATGCCAAA:

The characters represent the following nucleotides:

A: Adenosine T: Thymidine C: Cytidine G: Guanosine

The underlined nucleotides TAC are substituted nucleotides produced by site-directed mutation of the naturally-occurring Bst 320 polymerase. (As would be apparent to someone skilled in this art, this DNA sequence does not indicate the starting codon.)

The DNA polymerase may also be one that has a DNA sequence that is complementary to Bst 320 or the modified Bst 320 DNA sequence, for instance, DNA sequences that would hybridize to one of the above DNA sequences of under stringent conditions. As would be understood by someone skilled in the art, the DNA sequence also contemplates those that encode a peptide having these characteristics and properties (including degenerate DNA code).

The DNA sequences and amino acid sequences contemplated include allelic variations and mutations (for instance, adding or deleting nucleotide or amino acids, sequence recombination or replacement or alteration) which result in no substantive change in the function of the DNA polymerase or its characteristics. For instance, the DNA polymerases encompass non-critical substitutions of nucleotides or amino acids that would not change functionality (i.e., such as those changes caused by a transformant host cell). In addition, the invention is intended to include fusion proteins and muteins of the DNA polymerases.

The DNA sequences and amino acid sequences for the modified and ummodified DNA polymerases are also obtainable by, for instance, isolating and purifying DNA polymerase from a Bacillus stearothermophilus, or a bacterial strain otherwise derived from Bacillus stearothermophilus, or other mesophilic bacillus strains such as Bacillus caldotenax or Bacillus caldolyticus. The DNA polymerases obtained from these organisms may be easily modified using conventional DNA modification techniques to achieve the properties of high fidelity, high processivity, thermostability and reduction in fluorescent dye-labeled ddCTP and ddATP selective discrimination, as long as the unmodified amino acid sequences have leucine-glutamate-glutamate at positions corresponding respectively to positions 342-344 of Bst 320 DNA polymerase and phenylalanine at a position corresponding to position 422 of Bst 320 DNA polymerase. For instance, using the primers and methods of screening described herein, someone skilled in the art could isolate a DNA polymerase having the same properties and function from other strains.

In another preferred embodiment, a DNA polymerase is used which has highly stable enzymatic activity—for instance, stable enough to withstand drying-down processes yet remain viable for DNA sequencing. Such DNA polymerases are described by the inventors in U.S. patent application Ser. No. 09/735,677 (the contents of which is incorporated herein by reference in its entirety). These modified Bst DNA polymerases have increased stability properties, such that they can be freeze-dried or dried-down in cold temperatures, or stored in ready-to-use liquid reaction mixtures, for extended lengths of time (e.g., at least eight weeks) at room temperature without significant loss of its quality as a DNA polymerase for accurate incorporation of dNTPs and ddNTPs, or their analogs, onto the 3′ end of an extending primer upon reconstitution in solution. That is, upon reconstitution in solution and use in standard DNA sequencing there is no significant variability in the quality of sequences produced, when compared to control (e.g., non-freeze-dried or non-dried-down) DNA polymerase. Following freeze-drying or drying-down and subsequent reconstitution, these polymerases can be used in known DNA sequencing protocols to generate excellent quality DNA sequences. These DNA polymerases also demonstrate higher thermostability than the wild-type Bst DNA polymerases. For instance, these polymerases typically have a half-life of polymerase activity at 65° C. for about 16 minutes, which is roughly twice as long as the wild-type Bst DNA polymerase.

Throughout this disclosure, “HiFi Bst” or “Bst 320” DNA polymerase refers to the unmodified naturally occurring DNA polymerase having proofreading 3′-5′ exonuclease activity, either isolated from the cells of a strain designated no. 320 of Bacillus stearothermophilus or produced by overexpression of the gene encoding this naturally occurring DNA polymerase. (As noted above, this Bst strain no. 320 and DNA polymerase are described in U.S. Pat. Nos. 5,747,298 and 5,834,253.) “HiFi Bst-II” refers to the modified form of “HiFi Bst” DNA polymerase which has the ability to reduce selective discrimination against fluorescent dye-labeled ddCTP and ddATP. HiFi Bst-II is an example of one preferred embodiment of this invention. (This Bst strain and DNA polymerase are described in U.S. Pat. No. 6,165,765.) Bst-II also has sufficient stability to be dried-down or freeze-dried or stored in ready-to-use liquid reaction mixtures, at room temperature for an extended period of time (such as at least eight weeks), without significant loss of its quality as a DNA polymerase for accurate incorporation of dNTPs and ddNTPs, or their analogs, onto the 3′ end of an extending primer upon reconstitution in appropriate solution. (This Bst strain and DNA polymerase are described in copending U.S. patent application Ser. No. 09/735,677.)

Thus, in one embodiment of the methods of the invention, the invention contemplates a method for extending a primer (or a pair of primers) using an enzymatic cycle primer extension reaction at low cycling temperatures (that is, temperatures below about 80° C.). The reaction mixture composition that comprises between about 10% and about 20% (and preferably about 15%) (v/v) glycerol, ethylene glycol, or a mixture thereof. The reaction is run in the presence of a moderately thermostable DNA polymerase such as one of those described above. Ideally, the reaction is carried out under conditions that the cycle reaction temperature fluctuates between a melting temperature of about 70° C. and a cooling (or annealing) temperature of about 37° C., so that the DNA polymerase repeatedly extends the primer or pair of primers. The method may include the further step of repeating the cycle primer extension reaction, as many times as is desired.

In another embodiment, a PCR or PCR-like reaction may be run at low temperatures below 80° C. In this method, copies of a selected segment of a double-stranded DNA are amplified in the presence of a forward primer and a reverse primer (where both may be of various lengths) to the template by repeated heating and cooling (or annealing) cycles. The reaction mixture composition comprises between about 10% and about 20% (and preferably about 15%) (v/v) glycerol, ethylene glycol, or a mixture thereof, in the presence of one of the moderately thermostable DNA polymerases described above. The reaction is preferably carried out under conditions that the reaction temperature fluctuates between a melting temperature of about 70° C. and a cooling (or annealing) temperature of about 37° C., so that the DNA polymerase repeatedly extends the forward and reverse primers. The method may include the further step of repeating the reaction, as many times as is desired.

In a further embodiment, molecules of a single primer of various lengths are extended by a moderately thermostable DNA polymerase with specific nucleotide terminations in the presence of ddNTPs or their analogs for low-temperature cycle sequencing below about 80° C. The ddNTP analogs may be fluorescent dye-labeled so that each members of the ddNTPs may emit different wavelengths, as those used in automated dye-labeled terminator DNA cycle sequencing. Or instead, the sequencing primer will be labeled with four different dyes to be used in pairing with the corresponding unlabeled member of the ddNTPs for a modified Sanger reaction as in fluorescent dye-labeled primer DNA cycle sequencing technology. Or alternatively, the low-temperature cycle primer extension termination reaction can be used in the classic Sanger protocol with radioactive isotope-labeled dATP for manual direct sequencing of a small amount of DNA template without prior PCR amplification.

Another embodiment contemplates a method for extending the molecules of a single primer annealed to a single-stranded copy of the double-stranded DNA product amplified in vitro without prior isolation or purification for direct cycle sequencing. For instance, a diluted crude amplified reaction product (preferably generated with a low-temperature PCR reaction catalyzed by a moderately thermostable DNA polymerase as described herein) is used as template and mixed with an excess amount of a sequencing primer, the four standard ddNTP terminators (ddATP, ddGTP, ddTTP and ddCTP) fluorescently labeled (or their corresponding analogs fluorescently labeled), a moderately thermostable DNA polymerase (preferably one with a reduced innate selective discrimination against incorporation of a subset of dye-labeled ddNTPs), a suitable concentration of dNTPs (dATP, dGTP, dTTP and dCTP), and a composition comprising a buffer in a solution containing between about 10% and about 20% (preferably 15%) (v/v) of glycerol, ethylene glycol, or mixture thereof. A standard cycle primer extension reaction(s) may then be run at a temperature below 80° C. for a sufficient number of times to extend the sequencing primer molecules to desired varying lengths, which extended molecules will be terminated specifically by fluorescently labeled ddNTPs or their corresponding analogs. Preferably, the cycle reaction temperature fluctuates between a melting temperature of about 70° C. and a cooling/annealing temperature of about 37° C.

In one preferred embodiment, the method of sequencing a DNA strand may comprise the steps of:

i) hybridizing a primer to a DNA template to be sequenced; and

ii) extending the primer using one of the above-described DNA polymerases, in the presence of a solution containing between about 10% and about 20% (v/v) (preferably about 15% (v/v)) glycerol, ethylene glycol, or a mixture thereof, adequate amounts of the deoxynucleotide bases dATP, dGTP, dCTP and dTTP, and the four dideoxynucleotide terminators, or their analogs, whereby the cycle reaction temperature fluctuates between a melting temperature of about 70° C. and a cooling or annealing temperature of about 37° C., and under such conditions that the DNA strand is sequenced. Preferably one of the deoxynucleotides is radioisotope-labeled, or the primer molecules are fluorescent dye-labeled, and more preferably all are fluorescent dye-labeled.

In another embodiment, the invention entails a dry or liquid ready-to-use reaction mixture or kit suitable for use in a low-temperature cycle primer extension reaction at temperatures below about 80° C. This reaction mixture or kit comprises a moderately thermostable DNA polymerase (such as one of those described above) that is pre-mixed with at least one enzymatic DNA primer extension reaction component suitable for use in DNA amplification or for specific extension terminations with dideoxyribonucleotide analogs. The reaction mixture is preferably pre-distributed into microcentrifuge tubes or in multiple-well plates, such as, for instance, those that are suitable for large-scale automated PCR or for large-scale automated DNA sequencing. This ready-to-use reaction mixture or kit can be stored at room temperature between about 22° C. and about 25° C. for at least eight weeks without losing its specific polymerization activity for DNA primer amplification or extension terminations.

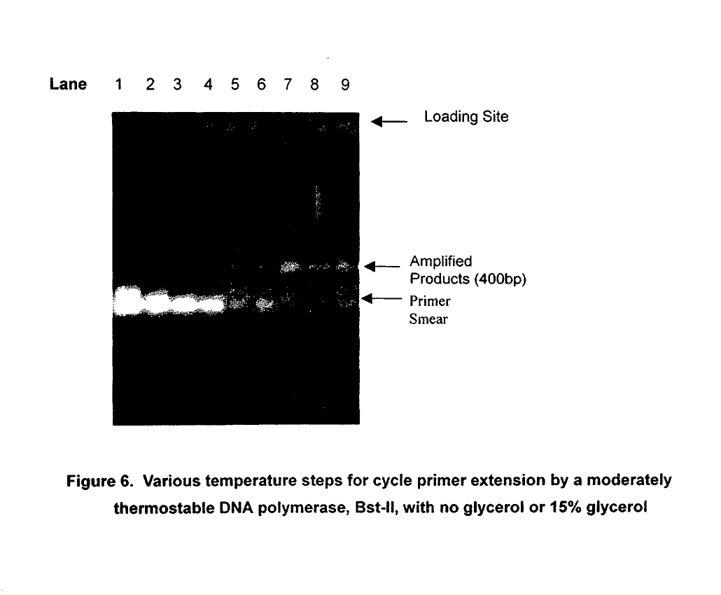

As an example of the present inventive methods, when a moderately thermostable DNA polymerase is used for low-temperature cycle primer extension, both annealing and primer extension can take place simultaneously at 45° C. Alternatively, 37° C. can be used as the annealing temperature and 45-50° C. the primer extension temperature. (See FIG. 6). Therefore, both the following protocol A and protocol B can be used for the low-temperature cycling steps with effective specific amplification:

(A) 70° C. 30 seconds and 45° C. 4 minutes for a total of 35 cycles; or

(B) 70° C. 30 seconds, 37° C. 20 seconds and 50° C. 3 minutes for a total of 35 cycles.

However, protocol B is preferred when the enzymatic primer extension is to generate reaction products with fluorescent dye-labeled ddNTP terminations for automated cycle sequencing. It is noted that the methods of this invention are not limited to either protocols A or B, but that these two protocols are exemplary of temperatures and cycles that work effectively with these methods. For instance, under certain circumstances, the extension time may be desired to be prolonged to about 11 minutes for long target segment amplification.

As another example, when the methods of this invention are used to generate amplification products for DNA cycle sequencing, a single primer in excess can be added to a reaction mixture containing 15% glycerol and a moderately thermostable DNA polymerase. The single-stranded primer oligonucleotides can then be extended to various lengths with specific nucleotide terminations in the presence of ddNTPs or their analogs, which may be fluorescently labeled. The template used for the cycle sequencing can be any purified double-stranded or single-stranded DNA fragments containing the target sequence, or an aliquot of the diluted amplification products derived from the low-temperature cycle-extended primer strands of the double-stranded DNA template described in this invention, without prior isolation and purification. Since the amplification products derived from the low-temperature cycle primer extension using a moderately thermostable DNA polymerase with high fidelity and high processivity as described in this invention are highly sequence-specific, prior isolation of the PCR product from the reaction mixture before being used as the template for DNA cycle sequencing is generally unnecessary.

The following non-limiting examples are illustrative of the invention.

### EXAMPLES

### Example 1

### The effect of Glycerol on 5′-3′ Polymerization Activity of Moderately Thermostable DNA Polymerases

The remaining activity of Bst-II DNA polymerase (produced according to U.S. Pat. No. 6,165,765) in the presence of different concentrations of Glycerol was determined as follows:

(a) In a series of numbered 0.5 ml microcentrifuge tubes were added the following:

| Tube No. | 1 | 2 | 3 | 4 | 5 | 6 | 7 |
|---|---|---|---|---|---|---|---|
| 5 × Reaction Buffer (ul) | 5 | 5 | 5 | 5 | 5 | 5 | 5 |
| DNTPs (1 mM each) | 1 | 1 | 1 | 1 | 1 | 1 | 1 |
| (ul) | | | | | | | |
| Calf Thymus DNA (ul) | 1 | 1 | 1 | 1 | 1 | 1 | 1 |

5 × Reaction Buffer (RB): 100 mM Tris-Cl, pH 8.5 containing 100 mM MgCl₂.
Calf Thymus DNA: DNase I activated, 1.5 ug/ul.

The mixtures above were firstly evaporated by Speed-Vacuum, then varying final concentrations of glycerol in each reaction mixture were achieved by adding an appropriate amount of a glycerol stock solution to the above microcentrifuge tubes as indicated below.

| Tube No. | 1 | 2 | 3 | 4 | 5 | 6 | 7 |
|---|---|---|---|---|---|---|---|
| Final Glycerol Conc. (v/ | 0 | 10% | 15% | 20% | 30% | 40% | 50% |
| v) | | | | | | | |
| α-³²P-dATP (ul) | 1 | 1 | 1 | 1 | 1 | 1 | 1 |
| Enzyme (0.36 ug/ul) (ul) | 1 | 1 | 1 | 1 | 1 | 1 | 1 |
| Glycerol (80% stock) (ul) | 0 | 3.1 | 5.0 | 6.9 | 10.6 | 14.4 | 18.1 |
| ddH₂O (ul) | 28 | 24.9 | 23.0 | 21.1 | 17.4 | 13.6 | 9.9 |
| Total Volume (ul) | 30 | 30 | 30 | 30 | 30 | 30 | 30 |

(α-³²P-dATP: Amersham, 800 Ci/mmol)

(b) All these tubes were incubated at 65° C. for 30 minutes. Then each of the reaction mixtures was pipetted onto a DE-81 filter. After all of the fluid has evaporated, the amount of radioactivity on each filter was measured with scintillation and recorded as X₁. Thereafter, the filters were washed three times with 0.3 M Na₂HPO₄ solution at room temperature, 10 minutes each times, dried at room temperature and then the amount of radioactivity on each filter was measured again and recorded as X₂. Incorporation ratio=X₂/X₁.

$$Remaining activity ( \% ) = \frac{\begin{matrix}Incorporation ratio of radioactivity in \\ different glycerol Concentration\end{matrix}}{Incorporation ratio of radioactivity without glycerol}$$

As shown in FIG. 1, a low concentration of glycerol that did not exceed 20% (v/v) increased the enzymatic activity of Bst-II DNA polymerase. However, at higher concentrations glycerol exhibited an inhibitory effect on the enzyme.

### Example 2

### The Effect of 40% Glycerol (v/v) on Low-temperature Cycle Primer Extension with Moderately Thermostable DNA Polymerases

Bst-II DNA polymerase was used for the study.

Template: pBluescript(+)

| | |
|---|---|
| Forward Primer: | 5′ GTAAAACGACGGCCAGT 3′ |
| Reverse Primer: | 5′ AACAGCTATGACCATG 3′ |

Experimental Procedure

(a) In a 0.2 ml microcentrifuge tube, were added the following

| | | |
|---|---|---|
| Template | 1 | (ul 16 ng/ul or 160 ng/ul) |
| Forward primer (10 pmol/ul) | 2.5 | ul |
| Reverse primer (10 pmol/ul) | 2.5 | ul |
| dNTPs (2.5 mM each) | 4 | ul |
| 5xRB | 5 | ul |

(b) The above mixture was firstly evaporated by Speed-Vacuum, then the following were added to the same microcentrifuge tube

| | | |
|---|---|---|
| ddH₂O | 11.75 | ul |
| 80% Glycerol | 11.25 | ul |
| Bst-II DNA polymerase (1 Unit/ul) | 2 | ul |

(c) The final mixture was subjected to the following temperature cycles.

| 70° C. | 30 sec |
|---|---|
| 45° C. | 4 min |
| 35 | cycles |

The reaction products were run on a 1% agarose gel for electrophoresis and stained by ethidium bromide.

The results illustrated in FIG. 2 show that in the presence of 40% glycerol as the reagent to lower the melting temperature of double-stranded DNA for cycle primer extension, non-specific amplification products of varying sizes were generated during the temperature cycling with either low or high concentration of template in the reaction mixture.

### Example 3

### The effect of Reduced Concentrations of Glycerol on Low-temperature Cycle Primer Extension with Moderately Thermostable DNA Polymerases

This experiment was designed to demonstrate that reduction of the concentration of glycerol to about 15% is useful for lowering the DNA melting temperature for specific cycle primer extension with moderately thermostable DNA polymerases

Materials and Methods

Bst-II DNA polymerase was used for the study.

Four different sets of templates and primers were selected representing varying lengths of the DNA segments to be amplified:

Template A. pBluescript(+) 10 ng/ul

| | |
|---|---|
| Forward Primer: | 5′ GTAAAACGACGGCCAGT 3′ |
| Reverse Primer: | 5′ AACAGCTATGACCATG 3′ |

Template B. A Rice genome BAC DNA 10 ng/ ul

| | |
|---|---|
| Forward Primer: | 5′ CTTAATTTAAGGTTCCGTG 3′ |
| Reverse Primer: | 5′ GCATTGGTAAGCAATGG 3′ |

Template C. A hybridization probe 50 ng/ul

| | |
|---|---|
| Forward Primer: | 5′ ACAAAGCACTGAACCTG 3′ |
| Reverse Primer: | 5′ TGGGACCTATCGTGTTG 3′ |

Template D. A subclone of BAC from rice genome 50 ng/ul

| | |
|---|---|
| Forward Primer: | 5′ CGAATTCCTGCAGCC 3′ |
| Reverse Primer: | 5′ GAACTAGTGGATCCCCC 3′ |

The low temperature cycle extension was carried out as follows:

(a) To a 0.2 ml microcentrifuge tube were added.

| | | | |
|---|---|---|---|
| Template | A, B, C or D | 1 | ul |
| Forward primer | A, B, C or D (10 pmol/ul) | 2.5 | ul |
| Reverse primer | A, B, C or D (10 pmol/ul) | 2.5 | ul |
| dNTP (2.5 mM each) | | 4 | ul |
| 5xRB | | 5 | ul |

(b) The mixture above was firstly evaporated by Speed-Vacuum, then the following were added to each microcentrifuge tube containing the evaporated reagents with different sets of template and primers to achieve a final concentration of 35% glycerol and 15% glycerol in the reaction mixture, respectively.

| | 1. with 35% Glycerol in mixture | 2. with 15% Glycerol in mixture |
|---|---|---|
| ddH₂O | 12.1 ul | 18.3 ul |
| 80% Glycerol | 10.9 ul | 4.7 ul |
| Polymerase (1U/ul) | 2 ul | 2 ul |

(c) All the microcentrifuge tubes with the reaction mixture were subjected to low temperature cycling as follows:

| 70° C. | 30 sec |
|---|---|
| 45° C. | 4 min |
| 35 | cycles total. |

The reaction products were run on a 1% agarose gel for electrophoresis and stained by ethidium bromide. The reaction products from the mixture containing 35% glycerol were loaded in lane 1, and the reaction products from the mixture containing 15% glycerol were loaded in lane 2.

The results illustrated in FIG. 3 show that, when a short segment of DNA of 250 bp or 400 bp long was the target product for cycle primer extension there were no amplification products produced at all during low temperature cycling, using 35% glycerol as the reagent for lowering the DNA melting temperature (FIG. 3 A1 and B 1). When longer target products, for example, 1 Kb and 2 Kb in length, were to be amplified under the identical conditions, enzymatic cycle primer extension was achieved with generation of both specific and non-specific amplification products when 35% of glycerol was used to lowering the melting temperature (FIG. 3 C1 and D1).

When a 15% glycerol was used as the reagent to lower the DNA melting temperature, specific amplification products ranging from 250 bp to 2 Kb in length were generated with a moderately thermostable DNA polymerase during low temperature cycling (FIG. 3 A2, B2, C2 and D2).

Based on the experimental results presented above, a low concentration of glycerol, for example at about 15% of final, in the reaction mixture has been adopted as the preferred reagent for lowering the DNA melting temperature in specific cycle primer extension by moderately thermostable DNA polymerases.

### Example 4

### Low-temperature Cycle Extension of DNA Primers of Different Lengths with Moderately Thermostable DNA Polymerases

In this example, the experiments were designed to demonstrate that the low-temperature cycle extension system with moderately thermostable DNA polymerases of this invention can be used for sequence-specific extension of primers of up to 30 base pairs in length.

The polymerases used were Bst-I (wild type produced according to U.S. Pat. No. 5,834,254), Bst-II, and Bca (TaKaRa Co.). The Klenow fragment (Sigma Chemical Co.) was used as a thermolabile DNA polymerase for comparison (Iakobashvili and Lapidot).

The template used was rice genome BAC B414f7.

The two pairs of primers used were:

A: 17mer forward primer:
5′TAG CTA TCT AAC TTA AT3′,
17mer reverse primer:
5′TTG TTT CTC TGA TGC AT3′,
B: 30mer forward primer:
5′TAG CTA TCT AAC TTA ATT TAA GGT TCC GTG3′,
30mer reverse primer:
5′TTG TTT CTC TGA TGC ATT GGT AAG CAA TGG3′.

The following reaction system (referred to hereafter as the Bst system) was used.

(a) In a 0.2 ml microcentrifuge tube were added:

| | | |
|---|---|---|
| Template (5 ng/ul) | 1 | ul |
| Forward primer (15 pmol/ul) | 2.5 | ul |
| Reverse primer (15 pmol/ul) | 2.5 | ul |
| dNTPs (2.5 mM each) | 4 | ul |
| 5xRB | 5 | ul |
| ddH₂O | 3.3 | ul |
| 80% Glycerol | 4.7 | ul |
| DNA polymerase (4U/ul) | 2 | ul |

(b) The microcentrifuge tubes were subjected to the following temperature cycling.

| | |
|---|---|
| 70° C. | 30 sec |
| 37° C. | 20 sec |
| 50° C. | 3 min |

35 cycles total.

The reaction products were run on a 1% agarose gel for electrophoresis and stained by ethidium bromide.

In addition, the method of using a reaction mixture containing 4.5 M proline and 17% glycerol in Tris-HCl buffer as recommended by Iakobashvili and Lapidot was also adopted for the reactions with Klenow fragment as the DNA polymerase (hereafter referred to as the Iakobashvili and Lapidot system). The reaction products were also run parallel to those obtained with the Bst system, and illustrated as follows in FIG. 4.

In FIG. 4, the following reaction products are shown in the respective lanes.

A: Reaction products with 17 mer primers:

A1: Klenow fragment using the Iakobashvili and Lapidot system.

A2: Klenow fragment with the Bst system.

A3: Bst-I polymerase with the Bst system.

A4: Bst-II polymerase with the Bst system.

A5: Bca polymerase with the Bst system.

B: Reaction products with 30 mer primers:

B1: Klenow fragment using the Iakobashvili and Lapidot system.

B2: Klenow fragment with the Bst system.

B3: Bst-I polymerase with the Bst system.

B4: Bst-II polymerase with the Bst system.

B5: Bca polymerase with the Bst system.

Molecular Ladders:

M1: λDNA/Hind III.

M2: DL 2,000 (from TaKaRa Co., with the DNA fragment of 2000, 1000, 750, 500, 250 and 100 bp respectively).

FIG. 4 shows that the moderately thermostable DNA polymerases, namely the natural form of Bst-I, the mutated Bst-II and Bca, all generated specific amplification products as a result of 17 mer primer extension (A3-A5) and of 30 mer primer extension (B3-B5) in the Bst system containing 15% glycerol in the reaction mixture as recommended for low temperature cycling. However, the thermolabile DNA polymerase, Klenow fragment, failed to produce a specific amplification product from 17 mer or 30 mer primer extension either in the Iakobashvili and Lapidot system (A1and B1) or in the Bst system (A2 and B2).

### Example 5

### High Fidelity Low-temperature Linear Cycle Sequencing with Bst-II DNA Polymerase in Stored Ready-to-use Reaction Pre-mixture

The current invention can be used to perform DNA sequencing with a genetically modified moderately thermostable DNA polymerase, Bst-II, to extend the primer over the GC-rich segments of the template which the commonly used heat-stable DNA polymerases with low processivity, such as ThermoSequenase™ or AmpliTaq™, are unable to overcome. Furthermore, all pre-measured ingredients of the reaction mixture with or without the primer pre-added can be pre-mixed and stored in individual microcentrifuge tubes or 96-well plates for at least eight (8) weeks at temperatures between 23° C. and 25° C.

Bst-II Cycle Sequencing Experiment

Bst-II was used as the DNA polymerase.

Template: bg08. This was a GC-rich segment of a subclone of rice genome BAC 129.

Primer: 5′ GAA TTG GAG CTC CAC CGC GG3′

Pre-mixed dye-ddNTPs: Optimized R6G-ddATP, ROX-ddCTP, TAMRA-ddUTP, and Bodipy F1-14-ddGTP, purchased from NEN™ Life Sciences Products.

(a) Into a 0.2 ml of microcentrifuge tube, the following ingredients were added

| | | |
|---|---|---|
| dNTPs (2.5 mM each) | 1 | ul |
| 5xRB | 5 | ul |
| Pre-mixed dye-ddNTPs | 4 | ul |
| Bst-II DNA polymerase (10U/ul) | 1 | ul |
| ddH₂O | 5.3 | ul |
| 80% Glycerol | 4.7 | ul |

The reaction pre-mixture in microcentrifuge tubes was stored at temperatures between 23° C. and 25° C. until use within eight (8) weeks.

(b) At the time of the experiment, 2.5 ul of template (150 ng/ul) and 1.5 ul of primer were added into a microcentrifuge tube containing the above pre-mixture.

(c) The contents in the microcentrifuge tube were mixed thoroughly and subjected to the following linear low temperature cycling.

70° C. for 30 sec,

37° C. for 20 ec,

45° C. for 3 min,

35 cycles total.

(d) Added 2.5 ul 3M NaOAc (pH5.2) and 55 ul 95% ethanol to each tube. The tube was inverted several times and then placed at room temperature for 20 min to precipitate the extension products.

(e) The mixture was centrifuged at 12,000 g for 20 min at room temperature.

(f) The supernatant was drawn off, and the pellet was rinsed with 120 ul 70% ethanol.

(g) Inverted the tube several times, placed the tube at room temperature for 15 min, and centrifuged the tube for 10 min at 12,000 g.

(h) The pellet was dried at 45° C., and resuspended in 1.2 ul loading buffer (5:1 of deionized formamide: 25 mM EDTA, pH8.0, with 50 mg/ml Blue Dextran).

(i) The sample was denatured at 95° C. for 3 min, then immediately placed on ice.

All of each sample was loaded onto 4.5% (6M urea) sequencing gel and the sequencing information was collected by an ABI PRISM™ 377 DNA Sequencer. The data were analyzed with the corresponding instrument (matrix) file.

For comparison, DNA sequencing of the identical template with the same primer was also performed, using two commercially available cycle sequencing kits, namely the DYEnamic™ ET terminator cycle sequencing kit with Thermo Sequenase™ (Amersham) and the ABI Prism™ BigDye™ Terminator cycle sequencing kit with AmpliTaq™ (ABI). The cycle sequencing procedures were carried out by following the protocols provided by the respective companies.

The results of the DNA sequencing were presented in FIG. 5 which shows the ABI Prism™ BigDye™ Terminator cycle sequencing kit with AmpliTaq™ (FIG. 5A) failed to accomplish efficient specific fluorescent dye-labeled ddNTP terminations during cycle primer extension over the GC-rich segment of the DNA template. In comparison, the Bst-II Cycle Sequencing system, even after the Bst-II DNA polymerase had been stored in a pre-mixed form for eight (8) weeks at 23-25° C., successfully overcame the GC-rich barrier in the template and generated adequate specific dye-labeled ddNTP terminations for DNA sequencing analyses (FIG. 5B). Similar to the ABI AmpliTaq™ kit, Thermo Sequenase™ used with the Amersham DYEnamic™ ET terminator cycle sequencing kit also failed to overcome the GC-rich segment of the template during the cycle primer extension reaction for automated fluorescent DNA sequencing (tracing not shown here).

In conclusion, the Bst-II Cycle Sequencing system which remains stable in ready-to-use pre-mixture at room temperature for at least eight (8) weeks is most suitable for large-scale high fidelity automated fluorescent DNA sequencing, especially when the templates contain GC-rich segments.

FIG. 5 shows DNA sequencing over a GC-rich segment, including a comparison of the performance of AmpliTaq™ in the ABI Prism™ BigDye™ Terminator cycle sequencing kit (A) with that of the Bst-II Cycle Sequencing System (B). FIGS. 5A and B represent two automated fluorescent DNA sequencing tracings of a GC-rich segment of the same template using the same prime for cycle extension. Both sequences were run in an ABI 377 sequencer. The shadowed zone illustrated in A represents the region out of quality control evaluated and reported by the computer.

A=generated with the AmpliTaq™ BigDye™ kit; B=generated with the Bst-II Cycle Sequencing system.

### Example 6

### Optimum Temperature Steps for Cycle Primer Extension with Moderately Thermostable DNA Polymerases

This experiment was designed to determine the optimum temperature steps for cycle primer extension with moderately thermostable DNA polymerases in a reaction mixture containing 15% glycerol as the agent to lower the DNA melting temperature.

Bst-II was the DNA polymerase used.

Template: A rice genome BAC DNA

| | |
|---|---|
| Forward Primer: | 5′ CTTAATTTAAGGTTCCGTG 3′ |
| Reverse Primer: | 5′ GCATTGGTAAGCAATGG 3′ |

(a) To each 0.2 ml microcentrifuge tube were added:

| | | |
|---|---|---|
| Template (10 ng/ul) | 1 | ul |
| Forward primer (10 pmol/ul) | 2.5 | ul |
| Reverse primer (10 pmol/ul) | 2.5 | ul |
| dNTPs (2.5 mM each) | 4 | ul |
| 5xRB | 5 | ul |

(b) The following were added to the microcentrifuge tubes to achieve:

| Final Concentration of Glycerol (v/v) | 0% | 15% | |
|---|---|---|---|
| ddH₂O | 8 ul | 3.3 | ul |
| 80% Glycerol | | 4.7 | ul |
| Bst-II DNA polymerase (1U/ul) | 2 ul | 2 | ul |

(c) The cycling temperature steps were as follows.

| Steps 1 | Steps 2 | Steps 3 | Steps 4 | Steps 5 |
|---|---|---|---|---|
| 70° C. 30 s | 70° C. 30 s | 70° C. 30 s | 70° C. 30 s | 70° C. 30 s |
| 37° C. 4 min | 37° C. 20 s | 37° C. 20 s | 37° C. 20 s | 45° C. 4 min |
| | 45° C. 3 min | 50° C. 3 min | 60° C. 3 min | |
| 35 cycles | 35 cycles | 35 cycles | 35 cycles | 35 cycles |

The reaction products were run on a 1% agarose gel for electrophoresis and stained by ethidium bromide. The results are illustrated in FIG. 6, in which, the lanes were loaded as follows:

1. No glycerol; 70° C. 30 s, 37° C. 4 min, 35 cycles.

2. No glycerol; 70° C. 30 s, 37° C. 20 s, 45° C. 3 min, 35 cycles.

3. No glycerol; 70° C. 30 s, 37° C. 20 s, 50° C. 3 min, 35 cycles.

4. No glycerol; 70° C. 30 s, 37° C. 20 s, 60° C. 3 min, 35 cycles.

5. 15% glycerol; 70° C. 30 s, 37° C. 4 min, 35 cycles.

6. 15% glycerol; 70° C. 30 s, 37° C. 20 s, 45° C. 3 min, 35 cycles.

7. 15% glycerol; 70° C. 30 s, 37° C. 20 s, 50° C. 3 min, 35 cycles.

8. 15% glycerol; 70° C. 30 s, 37° C. 20 s, 60° C. 3 min, 35 cycles.

9. 15% glycerol; 70° C. 30 s, 45° C. 4 min, 35 cycles.

The results in FIG. 6 show that the most effective cycle primer extension with moderately thermostable DNA polymerases, such as Bst-II, is obtained with a single annealing and extension temperature at 45° C. (Lane 9), or annealing at 37° C. and extension at 45° C.-50° C. in the presence of 15% glycerol used as the melting-temperature-lowering agent. Although both temperature cycling protocols of Steps 3 and Steps 5 can be used for specific primer extension in DNA amplification, the cycling protocol of Steps 3 with 70° C. 30 s, 37° C. 20 s, 50° C. 3 min, 35 cycles is preferred (Lane 7) when the enzymatic primer extension is used to generate reaction products with fluorescent dye-labeled ddNTP terminators for automated cycle DNA sequencing.

### Example 7

### Direct Low Temperature Cycle Sequencing of Amplified Products Generated by Moderately Thermostable DNA Polymerases in Stored Ready-to-use Reaction Pre-mixture

This example demonstrated that all pre-measured ingredients of the reaction mixture for low temperature primer extension, including a moderately thermostable DNA polymerase, with or without the primers pre-added can be pre-mixed and stored in individual microcentrifuge tubes or 96-well plates for at least eight (8) weeks at temperatures between 23° C. and 25° C. until use for the amplification reaction. In addition, the amplified reaction products can be used directly for automated DNA sequencing without prior purification.

Bst-II was used as the DNA polymerase.

Template: H525d9, a BAC of rice genome,

| | |
|---|---|
| Forward primer: | 5′ TTT CAG GGT CCC TTA TAT CTC 3′, |
| Reverse primer: | 5′ TCG CTT CTC CTC ATA ATC GAT 3′. |

Pre-mixed dye-ddNTPs: Optimized R6G-ddATP, ROX-ddCTP, TAMRA-ddUTP, and Bodipy F1-14-ddGTP, purchased from NEN™ Life Sciences Products.

(a) Into a 0.2 ml of microcentrifuge tube, the following ingredients were added:

| | | |
|---|---|---|
| Forward primer (10 pmol/ul) | 2 | ul |
| Reverse primer (10 pmol/ul) | 2 | ul |
| dNTPs (2.5 mM each) | 2 | ul |
| 5 x RB | 5 | ul |
| Bst-II DNA polymerase (10 U/ul) | 1 | ul |
| ddH₂O | 4.3 | ul |
| 80% Glycerol | 4.7 | ul |

The reaction pre-mixture in the microcentrifuge tube was stored at temperature between 23° C. and 25° C. until use within eight (8) weeks.

(b) At the time of experiment, the following were added to the stored pre-mixture:

| | | |
|---|---|---|
| Template (2.5 ng/ul) | 1 | ul |
| ddH₂O | 3 | ul |

(c) The ingredients in the microcentrifuge tube were thoroughly mixed and subjected to temperature cycling in the following protocol.

70 ° C. for 30 sec,

37 ° C. for 20 sec,

45° C. for 3 min,

35 cycles total.

(d) Cycle sequencing of the retrieved amplified products after purification.

(1) After the temperature cycling was completed, the reaction products were loaded onto a 1% low melting point agarose gel for electrophoresis.

(2) After electrophoresis, the target agarose blocks were cut out from the gel and weighed.

(3) To the cut-out agarose blocks, 0.04V of 25×Conc. Buffer (Roche) was added, and the mixture was incubated at 65° C. for 15 min to melt the gel.

(4) After additional incubation at 45° C. for 5 min, an appropriate amount of agarase(IU/ul, Roche) was added at IU/100 mg of agarose gel.

(5) After further digestion for 1 hour at 45° C., 1/10V of 3M NaOAc (pH5.2) was added. The tube was placed on ice for 15 min and then spun at 12,000 g and at 4° C. for 15 min.

(6) The supernatant was extracted with equal volume of phenol-chloroform twice, and of chloroform once. After each extraction, the mixture was centrifuged at 12,000 g for 5 min to collect the top aqueous layer.

(7) A 3V of 95% ethanol was added to the extracted aqueous phase; then the mixture was centrifuged at 12,000 g at 4° C. for 15 min after chill on ice for 15 min. The pellet was washed in 250 ul of 70% ethanol, dried and dissolved in 20 ul of ddH₂O.

(8) Sequencing the retrieved amplified products with the forward primer was performed as described above in 5 (c)-(k), using the high fidelity low-temperature linear cycle sequencing with Bst-II DNA polymerase in stored ready-to-use reaction pre-mixture.

Alternatively, an aliquot of the amplified products in the reaction mixture generated in step 7. (c) was sequenced with the Bst-II Sequencing System directly without retrieval and prior purification. The example of this direct cycle sequencing procedure is described as follows.

(1) Into a 0.2 ml of microcentrifuge tube, the following ingredients were added:

| | | |
|---|---|---|
| dNTPs (2.5 mM each) | 1 | ul |
| 5xRB | 5 | ul |
| Pre-mixed dye-ddNTPs (NEN ™) | 4 | ul |
| Bst-II DNA polymerase (10U/ul) | 1 | ul |
| ddH₂O | 5.3 | ul |
| 80% Glycerol | 4.7 | ul |

The reaction pre-mixture in microcentrifuge tubes was stored at temperatures between 23° C. and 24° C. until use within eight (8) weeks.

(2) At the time of experiment, the following were added into each microcentrifuge tube.

| | | |
|---|---|---|
| 1/20 diluted reaction product mixture [7.(c)] | 1 | ul |
| Forward primer (10 pmol/ul) | 1.5 | ul |
| ddH₂O | 1.5 | ul |

(3) The contents in the microcentrifuge tube were mixed thoroughly and subjected to the following linear low temperature cycling.

70° C. for 30 sec,

37° C. for 20 sec,

45° C. for 3 min,

35 cycles total.

The DNA in the reaction mixture was precipitated, washed and loaded onto sequencing gel for electrophoresis and analyzed as described above under Example 5 above. The sequencing tracings showed that the DNA sequences obtained by both methods were identical. They indicate that low temperature cycle primer extension with moderately thermostable DNA polymerases may generate highly specific amplified DNA products which can be used for direct sequencing without further isolation.

### REFERENCES

1. Sanger, F., Nicklen, S. & Coulson, A. R. Proc. Nat. Acad. Sci. USA 74: 5463-5467. 1977

2. Linda G. Lee, Charles R. Connell, Sam L. Woo, et al. Nucleic Acids Res. 20(10): 2471-2483. 1992

3. Molly Craxton. Methods: A Companion to Methods in Enzymology 3(1): 20-26. 1991

4. Hansneter Saluz, Jean-Pierre Jost. Proc. Nat. Acad. Sci. USA 86: 2602-2606. 1989

5. Ye, S. Y. & Hong, G. F., Scientia Sinica (Series B) 30: 503-506. 1987

6. Fuller, C. W.(1995) U.S. Pat. No. 5,432,065

7. Robert Iakobashvili, Aviva Lapidot. Nucleic Acids Res. 27(6): 1566-1568. 1999

8. U.S. Pat. No. 6,165,765, to Hong & Huang. DNA polymerase having high stability and ability to reduce innate selective discrimination against fluorescent dye-labeled dideoxynucleotides.

All references are incorporated by reference herein in their entirety.

What is claimed is:

1. A method for extending a primer or a pair of primers using an enzymatic cycle primer extension reaction at temperatures below about 80° C., comprising the step of mixing a template DNA with a primer or a pair of primers and a natural or a modified form of a moderately thermostable DNA polymerase from an organism selected from the group consisting of Bacillus stearothermophilus, Bacillus caldotenax and Bacillus caldolyticus, in a solution containing between about 10% and about 20% (v/v) glycerol, ethylene glycol, or a mixture thereof, under conditions that the cycle reaction temperature fluctuates between a melting temperature of about 70° C. and an annealing temperature of about 37° C., so that the DNA polymerase repeatedly extends the primer or pair of primers.

2. The method of claim 1, wherein the glycerol, ethylene glycol, or mixture thereof is present in about 15% (v/v).

3. The method of claim 1, wherein the DNA polymerase has an optimum enzymatic activity at about 65° C.

4. The method of claim 1, wherein the DNA polymerase has an amino acid sequence that shares not less than 95% homology of a DNA polymerase isolated from Bacillus stearothermophilus, Bacillus caldotenax or Bacillus caldolyticus.

5. The method of claim 1, which comprises the further step of repeating the cycle primer extension reaction.

6. The method of claim 1, wherein copies of a selected segment of a double-stranded DNA are amplified in the presence of a forward primer and a reverse primer to the template by repeated heating and cooling cycles.

7. The method of claim 6, wherein the forward primer and reverse primer may be of varying lengths.

8. The method of claim 1, wherein molecules of a single primer of various lengths are extended with specific nucleotide terminations in the presence of ddNTPs or their analogs for cycle sequencing.

9. A method for extending the molecules of a primer annealed to a DNA template for direct cycle sequencing of in vitro amplified double-stranded DNA products without prior isolation or purification, comprising the steps of:
(i) mixing diluted crude amplified reaction product with an excess amount of a sequencing primer, the four standard ddNTP terminators or their corresponding analogs, a native or modified form of a moderately thermostable DNA polymerase selected from the group consisting of Bacillus stearothermophilus, Bacillus caldotenax and Bacillus caldolyticus, a suitable concentration of dNTPs, and a composition comprising a buffer in a solution containing about 10% to about 20% of glycerol, ethylene glycol, or mixture thereof, and
(ii) effecting cycle primer extension reaction(s) at a temperature below 80° C. for a sufficient number of times to extend the sequencing primer molecules to desired lengths terminated specifically by ddNTPs or their corresponding analogs.

10. The method of claim 9, wherein in vitro amplified double-stranded DNA products are generated by extending a primer or a pair of primers using a enzymatic cycle primer extension reaction at temperatures below about 80° C., comprising the step of mixing a target segment of DNA with a primer or a pair of primers and a natural or a modified form of a moderately thermostable DNA polymerase from an organism selected from the group consisting of Bacillus stearothermophilus, Bacillus caldotenaxo and Bacillus caldolyticus, in a solution containing about 10% to about 20% (v/v) glycerol, ethylene glycol, or a mixture thereof, under conditions that the cycle reaction temperature fluctuates between a melting temperature of about 70° C. and a cooling temperature of about 37° C., so that the DNA polymerase repeatedly extends the primer or pair of primers.

11. The method of claim 9, wherein the moderately thermostable DNA polymerase has an amino acid sequence that shares not less than 95% homology of a DNA polymerase isolated from Bacillus stearothermophilus, Bacillus caldotenax or Bacillus caldolyticus.

12. A dry or liquid ready-to-use reaction mixture suitable for use in a low-temperature cycle primer extension reaction at temperatures below about 80° C., comprising a moderately thermostable, natural or modified DNA polymerase from an organism selected from the group consisting of Bacillus stearothermophilus, Bacillus caldotenax or Bacillus caldolyticus, that is pre-mixed with at least one enzymatic DNA primer extension reaction component suitable for use in DNA amplification or for specific extension terminations with dideoxyribonucleotide analogs.

13. The ready-to-use reaction mixture of claim 12, wherein the moderately thermostable DNA polymerase is a natural or modified DNA polymerase from an organism selected from the group consisting of Bacillus stearothermophilus, Bacillus caldotenax or Bacillus caldolyticus, or a moderately thermostable DNA polymerase which has an amino acid sequence that shares not less than 95% homology with a DNA polymerase isolated from Bacillus stearothermophilus, Bacillus caldotenax or Bacillus caldolyticus.

14. The ready-to-use reaction mixture in claim 12, which is pre-distributed into microcentrifuge tubes or in multiple-well plates.

15. The ready-to-use reaction mixture in claim 13, which is pre-distributed into microcentrifuge tubes or in multiple-well plates.

16. The ready-to-use reaction mixture of claim 14, which is pre-distributed into microcentrifuge tubes or in multiple-well plates, and remains stable at temperatures between 22° C. and 25° C. for at least eight weeks.

17. The ready-to-use reaction mixture of claim 15, which is pre-distributed into microcentrifuge tubes or in multiple-well plates, and remains stable at temperatures between 22° C. and 25° C. for at least eight weeks
